(19) United States
(12) Patent Application Publication
(10) Pub. No.: US 2020/0135341 A1
(43) Pub. Date: Apr. 30, 2020

(54) MULTI-CONTACT HEALTHCARE INFORMATION COLLECTION SYSTEM

(71) Applicant: Preferred Provider Holdco, LLC, Northbrook, IL (US)

(72) Inventor: Brad Morehead, Winnetka, IL (US)

(21) Appl. No.: 16/669,183

(22) Filed: Oct. 30, 2019

Related U.S. Application Data
(60) Provisional application No. 62/752,640, filed on Oct. 30, 2018.

(51) Int. Cl. G16H 80/00; G16H 10/60; G06Q 40/08

(52) U.S. Cl. CPC G16H 80/00; G06Q 40/08; G16H 10/60

(57) ABSTRACT

A method and apparatus receive an identifier and determining, from the identifier, a particular patient and a related party. The method and apparatus further determine a contact method for the related party and sending, to the related party via the contact method, a request for healthcare information for the particular patient. The method and apparatus even further comprise receiving the requested healthcare information from the related party.

### CROSS-REFERENCE TO RELATED APPLICATION

This application claims priority from U.S. Pat. App. Ser. No. 62/752,640 filed Oct. 30, 2018, entitled “Multi-Contact Healthcare Information Collection System”, the entire disclosure of which is hereby incorporated by reference in its entirety.

### BACKGROUND OF THE DISCLOSURE

### 1. Field of the Disclosure

The disclosure relates in general to healthcare information collection, and more particularly, to a multi-contact healthcare information collection, such as that performed by a healthcare provider.

### 2. Background Art

Healthcare patients may be a first-time patient or may switch healthcare providers. Healthcare providers must collect information from such patients. This information includes, but is not limited to, patient consents, Power of Attorney (PoA), primary care physician referral, health/medical records, patient payment information, and preferred pharmacy information.

In the case of healthcare patients, the ability to obtain patient information is a difficult, laborious, time-consuming manual and non-intuitive process. In particular, elderly patients, minor patients, patients with guardians, and/or non-self-responsible patients are patients that are particularly difficult to obtain patient information for. In many instances, an elderly person may have different individuals in charge of financial decisions, medical decisions, and scheduling decisions. It becomes difficult not only to obtain consent for treatment, for example, but to figure out and contact the party that is responsible for providing consent.

### SUMMARY OF THE DISCLOSURE

The disclosure is directed to a method that comprises receiving an identifier determining, from the identifier, a particular patient and a related party. The method further comprises determining a contact method for the related party and sending, to the related party via the contact method, a request for healthcare information for the particular patient. The method yet further comprises receiving the requested healthcare information from the related party.

The disclosure is also directed to an apparatus comprising a processor and a network interface. The processor receives an identifier, determine, from the identifier, a particular patient and a related party, and determining a contact method for the related party. The network interface sends, to the related party via the contact method, a request for healthcare information for the particular patient, and receive the requested healthcare information from the related party.

In some configurations, the network interface further transmits, to the related party, an invitation to a secure group chat session over the contact method.

In some configurations, the healthcare information includes at least one of patient consents, Power of Attorney (PoA), primary care physician referral, health/medical records, patient payment information, and preferred pharmacy information.

In some configurations, the patient payment information includes payor(s) for a particular patient and billing information.

In some configurations, the billing information includes an insurance company, insurance contact information, insurance enrollee identification number, and insurance group number.

In some configurations, the identifier is a unique identifier including at least one of a name, an address, and a social security number.

In some configurations, the method further comprises resolving any problems with the received identifier.

In some configurations, the related party is at least one of a parent, guardian, and spouse.

In some configurations, the contact method includes at least one of text, fax, audio, voice recognition, digital input, email, and voice call.

In some configurations, the method further comprises receiving, by the related party, an invitation to a secure group chat session over the contact method.

In some configurations, the secure group chat session is a virtual chat room.

In some configurations, the method further comprises resolving any problems with the received identifier if the inputted identifier does not match a known patient.

In some configurations, the method further comprises varying the contact method depending on at least one of a time and date.

In some configurations, the method further comprises identifying a preferred contact method that resulted in receiving the requested healthcare information, and recording the preferred contact method for a particular patient.

In some configurations, the contact method is one of a plurality of successful contact methods that result in receiving the requested healthcare information, the method further comprising at least one of choosing a preferred contact method from the plurality of successful contact methods and ranking the plurality of successful contact methods.

### BRIEF DESCRIPTION OF THE DRAWINGS

The disclosure will now be described with reference to the drawings wherein:

FIG. 1 illustrates an example healthcare information collection system, a version of which may comprise a control module, in accordance with the embodiments disclosed herein;

FIG. 2 illustrates an example schematic representation of a computing device, a version of which may comprise the control module, in accordance with the embodiments disclosed herein;

FIG. 3 illustrates an example flowchart for a typical sequence for collecting healthcare information, in accordance with the embodiments disclosed herein; and

FIG. 4 illustrates an example method of receiving healthcare information, in accordance with the embodiments disclosed herein.

### DETAILED DESCRIPTION OF THE DISCLOSURE

While this disclosure is susceptible of embodiment in many different forms, there is shown in the drawings and described herein in detail a specific embodiment(s) with the understanding that the present disclosure is to be considered as an exemplification and is not intended to be limited to the embodiment(s) illustrated.

It will be understood that like or analogous elements and/or components, referred to herein, may be identified throughout the drawings by like reference characters. In addition, it will be understood that the drawings are merely schematic representations of the invention, and some of the components may have been distorted from actual scale for purposes of pictorial clarity.

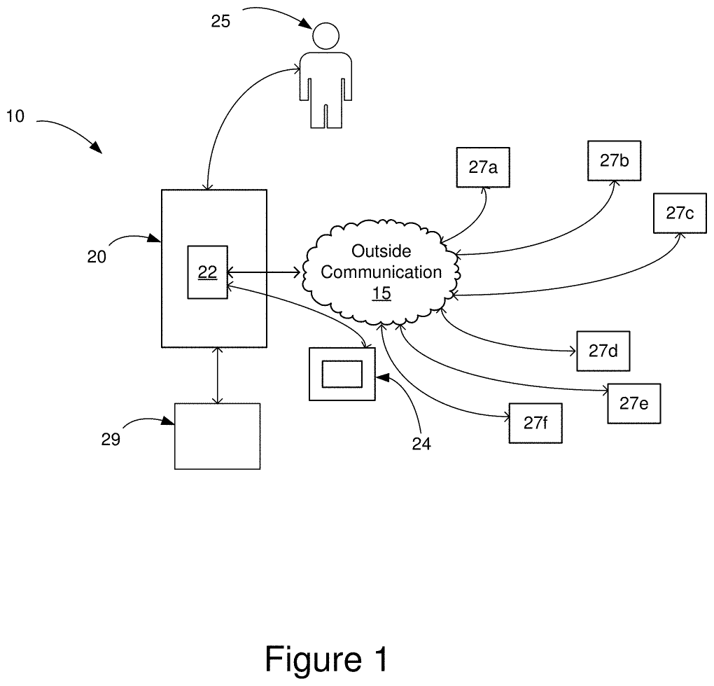

Referring now to the drawings and in particular to FIG. 1, a healthcare information collection system 10 is disclosed that includes a control module 20 that is comprised of a communication module 22. The communication module 22 is in communication with an outside communication network 15. The control module 20 communicates with one or more of user equipment (UE) 27a-f to collect the healthcare information described herein. The UE 27a-f can be a personal computer, a tablet computer, a smartphone, a non-smartphone, a fax machine, or any other device that a user can provide healthcare information to the control module 20. The healthcare information collection system 10 further includes a programming module 24.

The methods and systems disclosed herein are configured to work in association with a healthcare information collection system, such as the healthcare information collection system 10. The healthcare information collection system 10 includes a number of different components which work together. Of course, variations to the basic components, or combinations of the separate components are contemplated. Indeed, certain healthcare information collection systems may separate some of the components described below into discrete units, or may couple multiple components into a single component. However, it will be understood that typical healthcare information collection systems incorporate many if not all of the components that are identified.

The healthcare information collection system 10 provides for a centralized or decentralized inputting, storing, and cross-referencing of healthcare data to facilitate a multi-contact method of obtaining patient healthcare information in an automated manner. Such healthcare information includes, but is not limited to, patient consents, Power of Attorney (PoA), primary care physician referral, health/medical records, patient payment information, and preferred pharmacy information. Such patient payment information includes payor(s) for a particular patient and billing information, such as insurance company, insurance contact information, insurance enrollee identification number, insurance group number, etc. This method and the healthcare information collection system 10 automates communication and confirmation of this process by using a most efficient and effective set of tools and communications possible. In at least some embodiments, machine learning is used over time to improve the efficacy with which the healthcare information collection system 10 operates.

Information is provided to the control module 20 on a particular patient (e.g., elderly patients, minor patients, patients with guardians, non-self-responsible patients, or any other patient, particularly those that are difficult to obtain healthcare information for) via a unique identifier (e.g., name, address, social security number, etc.). This information is then cross-referenced with any existing information in the control module 20 to search for related data, a related party(ies) and a previous recommended communication pattern(s). If a preferred contact method is found, that path is used, if no path is found for that particular patient or related and responsible parties, then the control module 20 seeks the optimal method of communication for the new parties via various existing communication protocols. The control module 20 then communicates with one or more UE 27a-f associated with these new parties.

The healthcare information collection system 10 implements a contact method for unknown parties as a sequential or simultaneous blast of various methods, via the communication module 22. These various contact methods include, but are not limited to: text, fax, audio, voice recognition, digital input, email, voice call, or any other communication method that can be used to collect healthcare information. The healthcare information collection system 10 can select a particular method that produces an optimal result, as determined by a specific formula or algorithm that may be user-, client- or system-specific, and is then deemed to be the preferred contact method for future required communications.

The healthcare information collection system 10 utilizes patient identifiers and contact methods that are private, but shared across providers, patients, related parties, systems, and more, to optimize a speed and efficacy of communication. Furthermore, the healthcare information collection system 10 may receive updates via an immediate feedback loop for individual contacts within a chain of communication. Additional responses that are required or group responses from one or more of the UE 27a-f will result in each responsible or related party (e.g., parent, guardian, spouse, or any other related party that has healthcare information for a patient) receiving an invitation to a secure group chat session 29, such as a virtual chat room, that allows rapid flow of communication over the previously identified preferred contact method, such as a preferred channel. As an example, for group information, chat, text, fax and voice communication may be integrated to provide a fastest and most efficient means of communication between the UE 27a-f providing the group information. Such group information can be provided via the group chat session 29.

Generally, program modules include routines, programs, objects, components, data structures, and the like that perform particular tasks or implement particular abstract data types. Moreover, those skilled in the art will appreciate that the computing devices need not be limited to a specialized healthcare information collection system 10 control module (which may be highly proprietary), a conventional server computing racks or conventional personal computers, and include other computing configurations, including hand-held devices, multi-processor systems, microprocessor based or programmable consumer electronics, network PCs, minicomputers, mainframe computers, and the like. Similarly, the computing devices described herein need not be limited to a stand-alone computing device, as the mechanisms may also be practiced in distributed computing environments linked through the outside communications network 15. In a distributed computing environment, program modules may be located in both local and remote memory storage devices.

Advantageously, a user 25 of the healthcare information collection system 10 can set any number of simultaneously contacted individuals (and, it will be understood that a single individual may be contacted in any number of different forms), such as individuals using the UE 27a-f, and a response can be solicited from any one of the individuals and any one of the messages that have been sent. The user 25 can set the amount of time that one or more of the UE 27a-f are allowed to respond. Moreover, any number of contacts can be tried simultaneously or sequentially, in any number of different formats/methods (i.e., phone, text, email, etc.). It will also be understood, that, if desired, tiers of users can be contacted (if desired), wherein the messages are sent in waves for any particular desired reason.

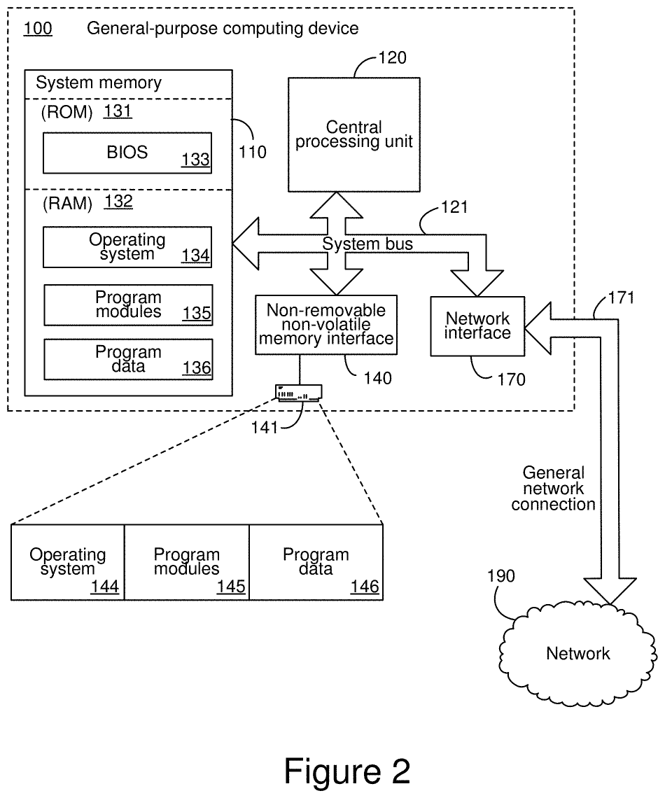

With reference to FIG. 2, an exemplary general-purpose computing device is illustrated in the form of the exemplary general-purpose computing device 100. The general-purpose computing device 100 may be of the type utilized for the control module 20 (FIG. 1) as well as the other computing devices with which control module 20 may communicate through outside communication network 15 (FIG. 1). As such, it will be described with the understanding that variations can be made thereto. The exemplary general-purpose computing device 100 can include, but is not limited to, one or more central processing units (CPUs) 120, a system memory 130 and a system bus 121 that couple various system components including the system memory to the processing unit 120. The processing unit 120 processes the information discussed herein for the control module 20 and the UE 27. The system bus 121 may be any of several types of bus structures including a memory bus or memory controller, a peripheral bus, and a local bus using any of a variety of bus architectures. Depending on the specific physical implementation, one or more of the CPUs 120, the system memory 130 and other components of the general-purpose computing device 100 can be physically co-located, such as on a single chip. In such a case, some or all of the system bus 121 can be nothing more than communicational pathways within a single chip structure and its illustration in FIG. 2 can be nothing more than notational convenience for the purpose of illustration.

The general-purpose computing device 100 also typically includes computer readable media, which can include any available media that can be accessed by computing device 100. By way of example, and not limitation, computer readable media may comprise computer storage media and communication media. Computer storage media includes media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by the general-purpose computing device 100. Communication media typically embodies computer readable instructions, data structures, program modules or other data in a modulated data signal such as a carrier wave or other transport mechanism and includes any information delivery media. By way of example, and not limitation, communication media includes wired media such as a wired network or direct-wired connection, and wireless media such as acoustic, RF, infrared and other wireless media. Combinations of the any of the above should also be included within the scope of computer readable media.

When using communication media, the general-purpose computing device 100 may operate in a networked environment via logical connections to one or more remote computers. The logical connection depicted in FIG. 2 is a general network connection 171 to the network 190, which can be a local area network (LAN), a wide area network (WAN) such as the Internet, or other networks. The computing device 100 is connected to the general network connection 171 through a network interface or adapter 170 that is, in turn, connected to the system bus 121. The network interface or adapter 170 transmits and receives the information disclosed herein as being transmitted to and received by the control module 20 and the UE 27. In a networked environment, program modules depicted relative to the general-purpose computing device 100, or portions or peripherals thereof, may be stored in the memory of one or more other computing devices that are communicatively coupled to the general-purpose computing device 100 through the general network connection 171. It will be appreciated that the network connections shown are exemplary and other means of establishing a communications link between computing devices may be used.

The general-purpose computing device 100 may also include other removable/non-removable, volatile/nonvolatile computer storage media. By way of example only, FIG. 2 illustrates a hard disk drive 141 that reads from or writes to non-removable, nonvolatile media. Other removable/non-removable, volatile/nonvolatile computer storage media that can be used with the exemplary computing device include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140.

The drives and their associated computer storage media discussed above and illustrated in FIG. 2, provide storage of computer readable instructions, data structures, program modules and other data for the general-purpose computing device 100. In FIG. 2, for example, hard disk drive 141 is illustrated as storing operating system 144, other program modules 145, and program data 146. Note that these components can either be the same as or different from operating system 134, other program modules 135 and program data 136. Operating system 144, other program modules 145 and program data 146 are given different numbers here to illustrate that, at a minimum, they are different copies.

With reference to FIG. 1, again, the foregoing description applies to the control module 20, as well as to any other computing devices in communication with the control module 20 through outside communication network 15, such as UE 27a-f. The control module 20 is coupled to the communication module 22. The communication module 22 facilitates outside communication in the form of voice and/or data. For example, the communication module may include a connection to a Plain Old Telephone Service (POTS) line, or a Voice-over-Internet Protocol (VOIP) line for voice communication. In addition, the communication module 22 may be configured to couple into an existing network, through wireless protocols (Bluetooth, 802.11a, ac, b, g, n, or the like) or through wired (Ethernet, or the like) connections, or through other more generic network connections. In still other configurations, a cellular link can be provided for both voice and data (i.e., GSM, CDMA or other, utilizing 2G, 3G, and/or 4G data structures and the like). The communication module 22 is not limited to any particular protocol or type of communication. It is, however, preferred that the communication module 22 be configured to transmit data bi-directionally, through at least one mode of communication. The more robust the structure of communication, the more manners in which to avoid a failure or a sabotage with respect to communication, such as to collect healthcare information in a timely manner.

The programming module 24 comprises a user interface which can configure the healthcare information collection system 10. In many instances, the programming module 24 comprises a keypad with a display that is connected through a wired connection with the control module 20. Of course, with the different communication protocols associated with the communication module 22, the programming module 24 may comprise a wireless device that communicates with the control module 20 through a wireless communication protocol (i.e., Bluetooth, RF, WIFI, etc.). In other embodiments, the programming module 24 may comprise a virtual programming module in the form of software that is on, for example, a smartphone, in communication with the communication module 22. In still other embodiments, such a virtual programming module may be located in the cloud (or web based), with access thereto through any number of different computing devices. Advantageously, with such a configuration, a user may be able to communicate with the healthcare information collection system 10 remotely, with the ability to change functionality.

Significantly, with the connectivity of the present control module 20 through the communication module 22, substantial additional and unique functionality can be provided. To leverage other data sources, the healthcare information collection system 10 can be configured to allow the user to connect the control module 20 with the user's social network account. For example, the healthcare information collection system 10 can be placed into communication with a user's social network (such as at least one of Facebook, Google+, and the like). Through such connectivity, the healthcare information collection system 10 has access to his or her friends and contacts within the social network and can use this data to, for example, prepare a call list to collect healthcare information. The healthcare information collection system 10 can select individuals from the social network, and the order of those individuals, that the user would like to be contacted in the event that healthcare information is collected. The healthcare information collection system 10 can select multiple individuals to be contacted simultaneously, or, alternatively, can sequentially contact individuals only if individuals higher on the list have not responded or received the message. As the social network includes the data for many of the contacts of the patient for which healthcare information is collected, the healthcare information collection system 10 can leverage the information that is already present in the user's social network.

Furthermore, the healthcare information collection system 10 can also tailor call lists based on a type of healthcare information being collected. For example, the healthcare information collection system 10 can select to use fax as a basis for communication when contacting a health care provider as healthcare providers typically have fax service. As another example, the healthcare information collection system 10 can select to use a voice call when contacting a party that is over a threshold age, as older persons typically use text and email less than younger parties, and vice versa.

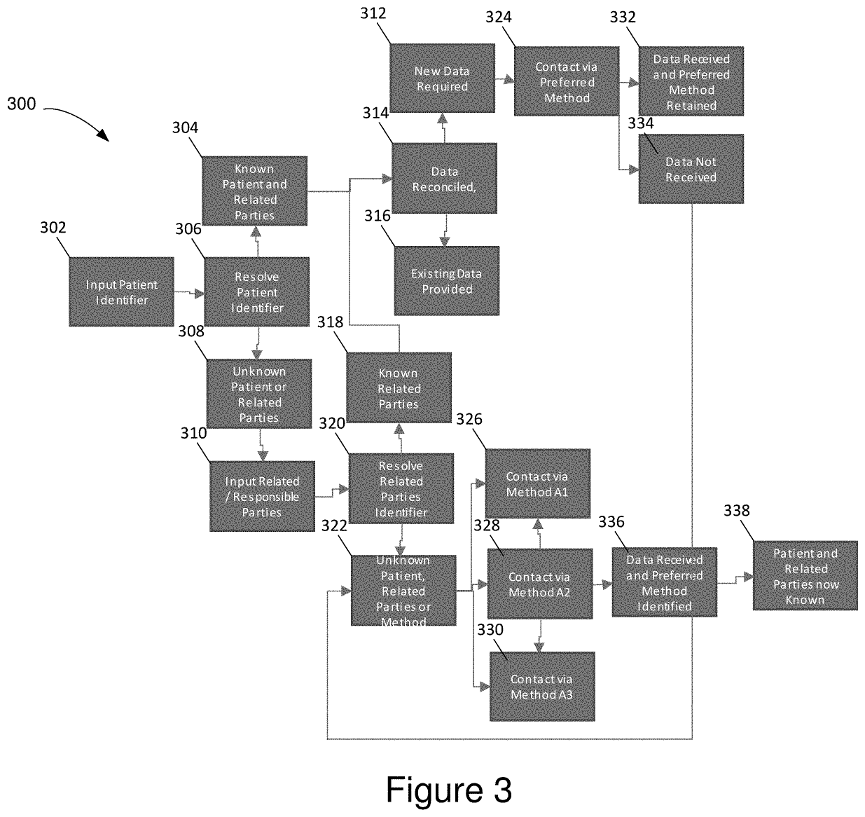

In another aspect of the disclosure, the healthcare information collection system 10 can assemble and take advantage of different call lists, and leverages these lists to improve the procedures that occur as a result of collecting healthcare information. A typical sequence for collecting healthcare information is shown as a flowchart 300 in FIG. 3.

With reference to FIG. 3, when healthcare information for a particular patient needs to be collected, an identifier for a particular patient is input at 302. For example, the user 25 can input the identifier in the control module 20 via the programming module 24. If the inputted identifier does not match a known patient, the control module 20 resolves any problems with the inputted identifier at 306. Otherwise, the control module 20 attempts to gather the required healthcare information with the identifier being associated with a known patient and related party(ies) at 304.

The control module 20 can determine if healthcare information for the particular patient is incomplete, that is new additional data is required for the particular patient, and, performs reconciliation of healthcare information for the particular patient at 314. In the event that the data is provided, the system determines that existing data for the particular patient has been provided at 316.

On the other hand, should the new additional data be required for the particular patient at 312, the control module 20 contacts, that is sends a request for healthcare information for the particular patient, one or more of the UE 27a-f via a preferred contact method at 324, as discussed herein. It will be understood that the preferred method may comprise one or more methods of communication, and, in some instances, such preferred methods may be varied depending on time, date, etc. Once the requested healthcare information is received by the control module 20, the control module 20 identifies the preferred contact method that resulted in receiving the required (requested) healthcare information at 332. The control module 20 then records the preferred contact method for the particular patient has been retained (or may modify the preferred contact method based on the information received). It will be understood that the particular patient and related contact party(ies) are now known at 338 where the information is determined initially.

Otherwise, the control module 20 determines that the requested information has not been received at 334, and the particular patient is treated as an unknown patient, along with their related party(ies) or contact method(s) at 322. In that instance, the system goes to the step 322 (described below), as either the patient is unknown, or the contact method is incorrect. In either case, necessary the information is not received.

Should the control module 20 not be able to resolve any problems with an identifier at 306, the identifier is determined to not be previously stored by the control module 20, that is the particular patient is unknown or the related party(ies) are unknown at 308. Additionally, should the requested information not be received at 334, as discussed above, the identifier is treated as a non-resolved problem identifier.

In either instance, the user 25 then inputs any known data for the particular patient and any responsible parties for the patient at 310, and that newly entered data becomes associated with the previously non-stored identifier. This newly input data is stored by the control module 20. The control module 20 attempts to resolve association of related party(ies) with the identifier at 320. If the control module 20 determines that the identifier has known related party(ies) at 318, 318 proceeds to 314. Otherwise, the control module 320 determines that the particular patient is unknown, the related party(ies) are unknown, or a method of contact is unknown at 322 (the same point as explained above where at 334, the data is not received).

The control module 20 then contacts the newly entered contact(s) via one or more of a method A1, method A2, and a method A3, at 326, 328, and 330, respectively, in an attempt to retrieve the unknown information from 322. Such methods, which can be different types of methods, are discussed above and can be sent simultaneously, sequentially, or some combination of both. The system then waits for a response to the contact from one or more of the methods. When the control module 20 receives the healthcare information the system can identify one or more preferred contact method(s) of obtaining such healthcare information at 336. Again, at this time, the system can determine that there are multiple contact methods that are successful and from there one can be chosen, they can be ranked, or they can all be utilized. The control module 20 determines that the particular patient and related contact party(ies) are now known at 338. Once known, the system can recognize the patient.

The system 10 can have additional functionality to amend or to process changes that may occur to the patient, the contact methods, etc., beyond that which is provided herein. The system 10 herein provides a health care provider with the necessary permissions and other information as necessary without the laborious process of calling, faxing, texting, emailing a number of different people to achieve the necessary authorizations. And, the system 10 can maintain these relationships so that future permission can be obtained quickly and with minimal time and disruption.

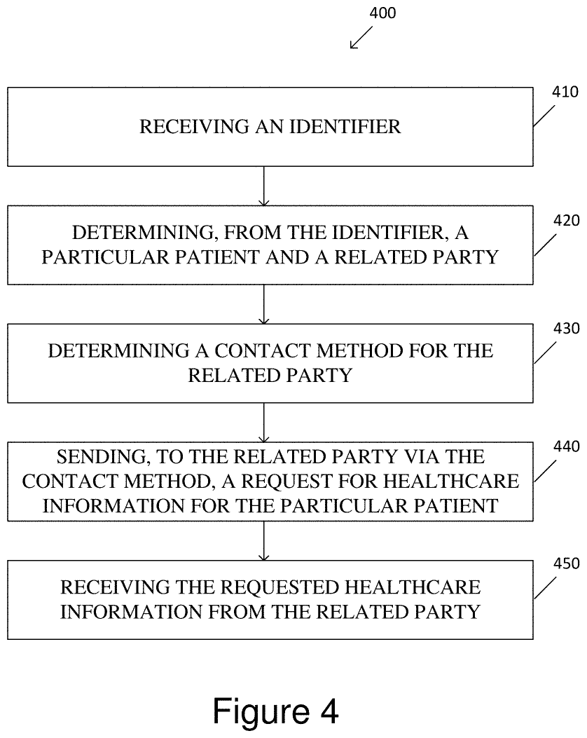

Referring now to FIG. 4, a method 400 of receiving healthcare information is disclosed. In at least one embodiment, the method 400 can be executed by the general-purpose computing device 100 shown in FIG. 2. The method 400 can begin at process 410 that includes receiving an identifier. For example, the process 420 can implement a Graphic User Interface (GUI) to provide a user with one or more data entry boxes in which the user 25 can enter the identifier. Process 410 can proceed to process 420.

Process 420 can include determining, from the identifier, a particular patient and a related party. In at least one embodiment, the related party includes at least one of a parent, guardian, spouse, or any other party that can provide the healthcare information discussed herein. In at least embodiment, process 420 can search one or more databases (not shown) for such information. Process 420 can proceed to process 430.

Process 430 can include determining a contact method for the related party. In at least one embodiment, the contact method includes at least one of text, fax, audio, voice recognition, digital input, email, voice call, or any other contact method that can be used to gather healthcare information from the related party. In at least one embodiment, process 430 can search one or more databases (not shown) for such information as being associated with the related party. Process 430 can proceed to process 440.

Process 440 can include sending (e.g., by the network interface or adapter 170), to the related party via the contact method, a request for healthcare information for the particular patient. In at least one embodiment, the healthcare information includes at least one of patient consents, Power of Attorney (PoA), primary care physician referral, health/medical records, patient payment information, preferred pharmacy information, or any other healthcare information that can be useful for a healthcare provider. Process 440 can proceed to process 450.

Process 450 can include receiving (e.g., by the network interface or adapter 170) the requested healthcare information from the related party. In at least one embodiment, the method 400 can end with process 450. In at least one embodiment, the method 400 further includes any of the features described above for FIGS. 1-3.

The foregoing description merely explains and illustrates the disclosure and the disclosure is not limited thereto except insofar as the appended claims are so limited, as those skilled in the art who have the disclosure before them will be able to make modifications without departing from the scope of the disclosure.

What is claimed is:

1. A method, comprising:
receiving an identifier;
determining, from the identifier, a particular patient and a related party;
determining a contact method for the related party;
sending, to the related party via the contact method, a request for healthcare information for the particular patient; and
receiving the requested healthcare information from the related party.

2. The method according to claim 1, wherein the healthcare information includes at least one of patient consents, Power of Attorney (PoA), primary care physician referral, health/medical records, patient payment information, and preferred pharmacy information.

3. The method according to claim 2, wherein the patient payment information includes payor(s) for a particular patient and billing information.

4. The method according to claim 3, wherein the billing information includes an insurance company, insurance contact information, insurance enrollee identification number, and insurance group number.

5. The method according to claim 1, wherein the identifier is a unique identifier including at least one of a name, an address, and a social security number.

6. The method according to claim 1, further comprising resolving any problems with the received identifier.

7. The method according to claim 1, wherein the related party is at least one of a parent, guardian, and spouse.

8. The method according to claim 1, wherein the contact method includes at least one of text, fax, audio, voice recognition, digital input, email, and voice call.

9. The method according to claim 1, further comprising receiving, by the related party, an invitation to a secure group chat session over the contact method.

10. The method according to claim 9, wherein the secure group chat session is a virtual chat room.

11. The method according to claim 1, further comprising resolving any problems with the received identifier if the inputted identifier does not match a known patient.

12. The method according to claim 1, further comprising varying the contact method depending on at least one of a time and date.

13. The method according to claim 1, further comprising:
identifying a preferred contact method that resulted in receiving the requested healthcare information; and
recording the preferred contact method for a particular patient.

14. The method according to claim 1, wherein the contact method is one of a plurality of successful contact methods that result in receiving the requested healthcare information, the method further comprising at least one of choosing a preferred contact method from the plurality of successful contact methods and ranking the plurality of successful contact methods.

15. An apparatus, comprising:
a processor to receive an identifier, determine, from the identifier, a particular patient and a related party, and determining a contact method for the related party; and
a network interface to send, to the related party via the contact method, a request for healthcare information for the particular patient, and receive the requested healthcare information from the related party.

16. The apparatus according to claim 15, wherein the healthcare information includes at least one of patient consents, Power of Attorney (PoA), primary care physician referral, health/medical records, patient payment information, and preferred pharmacy information.

17. The apparatus according to claim 16, wherein the patient payment information includes payor(s) for a particular patient and billing information.

18. The apparatus according to claim 17, wherein the billing information includes an insurance company, insurance contact information, insurance enrollee identification number, and insurance group number.

19. The apparatus according to claim 15, wherein the identifier is a unique identifier including at least one of a name, an address, and a social security number.

20. The apparatus according to claim 15, wherein the related party is at least one of a parent, guardian, and spouse.

21. The apparatus according to claim 15, wherein the contact method includes at least one of text, fax, audio, voice recognition, digital input, email, and voice call.

22. The apparatus according to claim 15, wherein the network interface further transmits, to the related party, an invitation to a secure group chat session over the contact method.

23. The apparatus according to claim 22, wherein the secure group chat session is a virtual chat room.
